(19) United States
(12) Patent Application Publication
(10) Pub. No.: US 2009/0173354 A1
(43) Pub. Date: Jul. 9, 2009

(54) Removably attachable retractable strap device

(76) Inventor: Yaakov Gold, Brooklyn, NY (US)

Correspondence Address: Esther E. Schwartz, 1898 Ocean Avenue, Brooklyn, NY, 11230, US

(21) Appl. No.: 12/006,904

(22) Filed: Jan. 7, 2008

(51) Int. Cl. A61F 5/37

(52) U.S. Cl. 128/876

(57) ABSTRACT

I have invented a removably attachable retractable strap device for use with conventional spinal backboards. The present invention essentially comprises a retractable strap device contained in a housing with a curved bottom to be removably attached to traditional spinal backboards for use in securing and transporting patients. The retractable strap is wound around a spring loaded retracting mechanism which is contained within a strap housing with a curved bottom. The strap housing is easily secured to a traditional spinal backboard from underneath the backboard with a spring-loaded clip mechanism so that the backboard rests on the curved bottoms of the housing devices. The spring-loaded clip is engaged by squeezing the squeeze plates, inserting the clip through a handhold or opening in the backboard, and then releasing the squeeze plates. In the preferred embodiment, the strap can be locked into an extended position by a locking mechanism so that a secure fit around the patient may be achieved. The preferred embodiment further provides a release mechanism for releasing the strap.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISC

Not Applicable

### BACKGROUND OF THE INVENTION

(1) Field of the Invention

This invention relates to strap devices for use in connection with spinal immobilization backboard devices. More particularly, this invention relates to a retractable strap housing designed to be removably attached to conventional spinal immobilization backboards with a quick release spring-loaded clip.

(2) Description of the Related Art Including Information Disclosed Under 37 CFR 1.97 and 1.98.

The current state of the art is filled with spinal backboards and various first aid equipment geared toward pre-hospital emergency care. Yet despite all the improvements in first aid equipment, many key difficulties with current spinal backboards have never been addressed or solved in an easily implemented, efficient, or cost-effective manner.

The role of the pre-hospital care provider, or an emergency medical technician (EMT), is to stabilize an injured or sick patient and then transport them to a medical facility, such as a hospital emergency room or specialty treatment center.

The United States Department of Transportation has created a National Standard Curriculum upon which most emergency medical technician and paramedic protocols courses and training are based.

When treating patients who have sustained trauma, the first issue considered is usually the ‘mechanism of injury.’ The rescuer, can use the events that caused the trauma (mechanism) to determine what steps need to be taken to correctly identify problems and to efficiently stabilize the patient for transport to an emergency room.

Of particular concern are injuries that affect, or potentially affect, the spine of the injured patient. The spinal cord is a major part of the central nervous system which conducts sensory and motor nerve impulses to and from the brain.

Injuries to the spinal column, or spinal cord, can result in serious or devastating permanent disabilities. Injuries to the spine can result in irreversible paralysis, loss of control over involuntary muscle function such as breathing, or death.

Where the mechanism of injury suggests possible spinal injury, such as a car accident, a fall from significant height, gunshot wounds, or a sports injury, the emergency medical technician will follow the accepted protocol of spinal care.

The most essential element of proper spinal care after injury is immobilization of the spine so as not to increase or aggravate injury to the spine. This immobilization is effected by applying a rigid cervical collar to immobilize the neck portion of the spine, and strapping the patient to a long board, known as a spine board, to keep the patient's spine from moving or shifting during transport.

Yet the current most widely used method of securing a patient to a spine board presents many problems and difficulties.

The method, essentially, comprises extending and straightening the patient's limbs so that the patient is lying in a supine position, or flat on his/her back. The patient is then rolled as a unit onto his/her side, while the emergency medical technician places a spine board on its side parallel to the patient. The patient is then rolled back onto the board, and secured to the board via a series of torso straps and wide tape that is used over the forehead and collar to secure the head.

While in theory this method should be effective, it is nearly impossible to execute fluidly. The first problem involves the straps used to secure the patient to the spine board. The straps must be attached to the spine board before the patient is placed on the board as the patients bulk will usually cover the holes to which the straps are typically affixed. When the board is placed on its side, the straps inevitably wind up stuck under the edge of the board. To secure the patient with the straps, the emergency medical technician must shift the board while the patient is on the board in order to free the straps. In addition to making the emergency medical technician's job more difficult, shifting the board causes the patient to shift, possibly compromising the spine.

The second problem posed is caused by the flat shape of the standard spine boards in use today. The flat shape of the board requires the board to lay flat against the ground while the patient is being secured. When the emergency medical technician tapes the patient's head into place, the board must be lifted somewhat to allow the technician to properly apply the tape. In addition, when emergency medical technicians are carrying a spine board and place it on a flat surface such as a stretcher, gurney, or the x-ray table at the emergency room, they tend to place it down a little too roughly to avoid crushing their fingers between the board and the surface. This further unnecessarily shifts and jostles the board, unnecessarily further endangering the patient's spine, and could in effect counteract all the benefits the spine board is supposed to provide.

The instant invention, a removably attachable retractable spinal backboard strap device, solves both those problems. The strap device of the instant invention is fully compatible with existing spine boards, and thus can easily be put into use.

Spinal backboards in the current art have a plurality of openings around the perimeter of the board to secure a plurality of straps to the spinal backboard. A significant portion of spinal backboards currently in use further provide a metal pin across each opening to latch a speed-clip to. Straps with a speed-clip at the end can therefore be easily latched to the pin provided in the opening. The novel strap device of the instant invention is compatible with all known spinal backboards in the current art, both with metal speed-clip pins, and without.

The novel strap device of the instant invention is easily attached and detached from a traditional spine board with novel squeeze and release plates without interfering with the metal pin provided in many spinal backboard openings, provides retractable straps eliminating tangling and trapping, raises a traditional spine board on curved feet to facilitate gently rolling the patient onto the board with minimal jostling, raises a traditional spine board to allow room for fingers to slip under the board, raises a traditional spine board to allow for ease in applying tape to secure patients head, and does not interfere with patient comfort nor does it block portions of the backboard that are required to be xray translucent.

There is currently no strap device in the prior art that is easily attached and detached from a traditional spine board, provides retractable straps eliminating tangling and trapping, raises a traditional spine board on curved feet to facilitate gently rolling the patient onto the board with minimal jostling, raises a traditional spine board to allow room for fingers to slip under the board, and does not interfere with patient comfort.

There are several types of devices in the prior art designed to facilitate patient spinal immobilization and transport. There are also devices in the prior art that provide various forms of retractable straps. However, none of these devices disclose the instant invention.

For example, U.S. Pat. No. 5,492,285 discloses a medical stretcher with retractable straps. However, the straps are permanently mounted to a medical stretcher or wheeled emergency table and cannot be used in connection with spine boards.

U.S. Pat. No. 4,699,132 discloses a patient restraint device attempting to address some of the same concerns as the instant invention. This device provides straps that fold in a complicated configuration that must be re-folded and secured each time. The straps can be attached anywhere to the spine board. However, this device does not address the concerns presented to the integrity of the patient's spine by the flat bottom of the traditional spine board when being transported by medical technicians during a rescue operation. Furthermore, this device does not disclose the retracting straps or the spring-loaded squeeze and release attachment means of the instant invention. Consequently, this device cannot be used to achieve the same results and perform the same functions as those provided by the instant invention. And lastly, the complicated folding structure makes it less practical and convenient.

Similarly, all other spine board strap-attachment means in the prior art do not contain all the novel improvements and features of the instant invention.

It is therefore an object of the present invention to provide a removably attachable retractable strap for securing a patient to a spine board.

It is a further object of the present invention to provide a squeeze and release spring loaded attachment means for attaching a retractable patient strap to a spine board. This method will allow the device to be easily applied to the backboard at various heights so that the strap can be in place at the portion of the patients torso that requires it.

It is a further object of the present invention to provide a removably attachable device for raising a spine board on curved feet to facilitate seamless transport of a patient without compromising the patient's spinal column during securement to the spine board, lifting, and setting down the spine board.

It is a further object of the present invention to provide a device that addresses the common concerns with traditional spine board use in rescue operations by providing spine board straps in a retractable housing to avoid entanglement and provide for quicker and easier patient securement, spring-loaded squeeze and release attachment means compatible with existing spine boards for simpler, quicker, and easier patient securement, and a retractable strap housing with a curved surface to raise the entire spine board on curved feet allowing easy lifting by medical personnel by providing space for fingers to slip under the board and further allowing the board to be gently tilted on the curved surface for seamless placement of the injured patient onto the spine board from another surface without undue jostling or possibly further compromising the patient's spine.

It is a further object of the present invention to address the common concerns with traditional spine board use with a removably attachable strap device with curved feet that is quicker and simpler to use, more convenient, more compact, and safer than traditional spine board strap devices in the current art.

It is a further object of the present invention to provide a spring-loaded squeeze and release mechanism for removably attaching straps to a spinal backboard.

It is a further object of the present invention to provide a spring-loaded squeeze and release mechanism for removably attaching retractable straps to a spinal backboard.

It is a further object of the present invention to provide a spring-loaded squeeze and release mechanism for removably attaching medical accessories to a spinal backboard.

It is a further object of the present invention to provide a spring-loaded squeeze and release mechanism for removably attaching medical accessories to a spinal backboard that is compatible with most existing spinal backboards and is economical and practical for widespread use.

### BRIEF SUMMARY OF THE INVENTION

We have invented a removably attachable retractable strap device for use with conventional spinal backboards. The present invention essentially comprises a retractable strap device contained in a housing with a curved bottom to be removably attached to traditional spinal backboards for use in securing and transporting patients. The retractable strap is wound around a spring loaded retracting mechanism which is contained within a strap housing with a curved bottom. The strap housing is easily secured to a traditional spinal backboard from underneath the backboard with a spring-loaded clip mechanism so that the combined weight of the backboard and the patient rests on the curved bottoms of the housing devices. The spring-loaded clip is engaged by squeezing the squeeze plates, inserting the clip through a handhold or opening in the backboard, and then releasing the squeeze plates. In the preferred embodiment, the strap can be locked into an extended position by a locking mechanism so that a secure fit around the patient may be achieved. The preferred embodiment further provides a release mechanism for releasing the strap.

### BRIEF DESCRIPTION OF THE SEVERAL VIEWS OF THE DRAWINGS

FIG. 1. is a perspective view of the removably attachable retractable strap device.

FIG. 2. is a side view of the removably attachable retractable strap device.

FIG. 3. is a perspective view of the removably attachable retractable strap device attached to a spinal backboard.

FIG. 4. is a side view of a spinal backboard illustrating how the removably attachable retractable strap device is inserted through an opening in the spinal backboard.

### DETAILED DESCRIPTION OF THE INVENTION

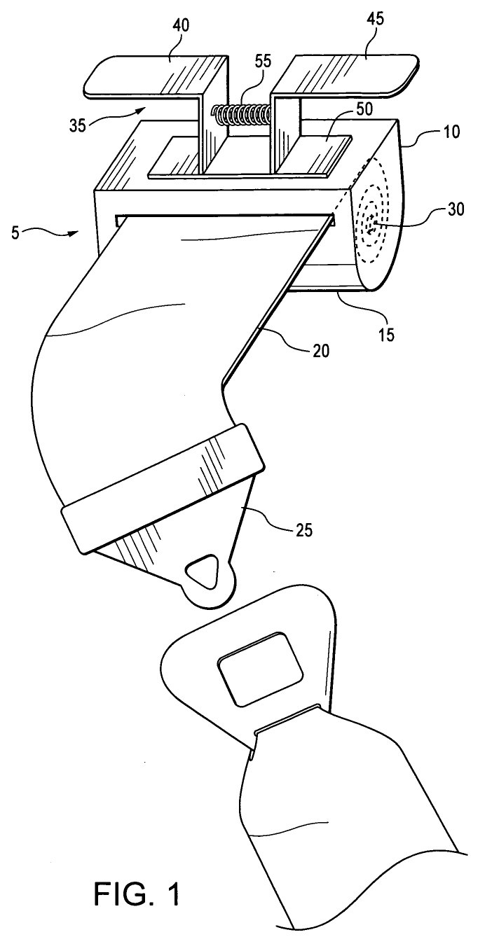

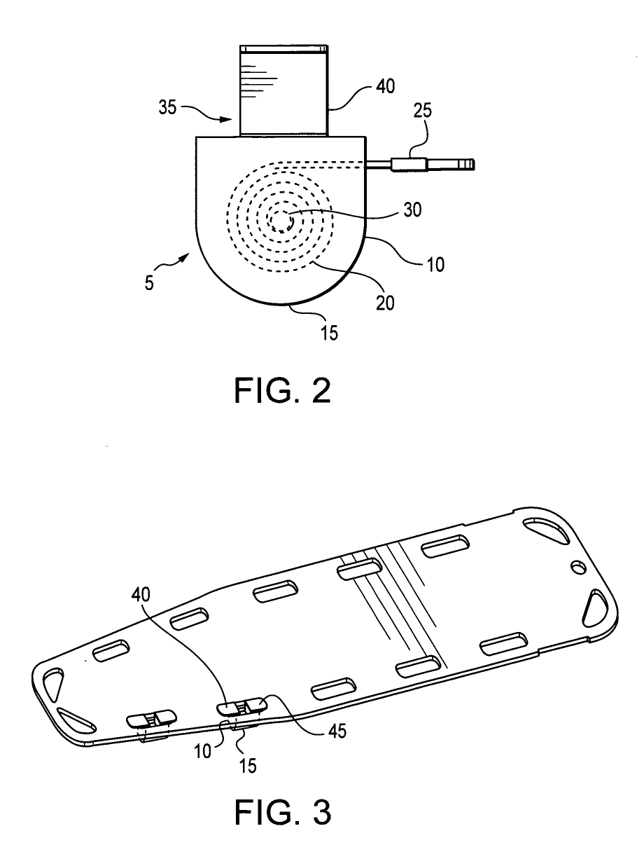

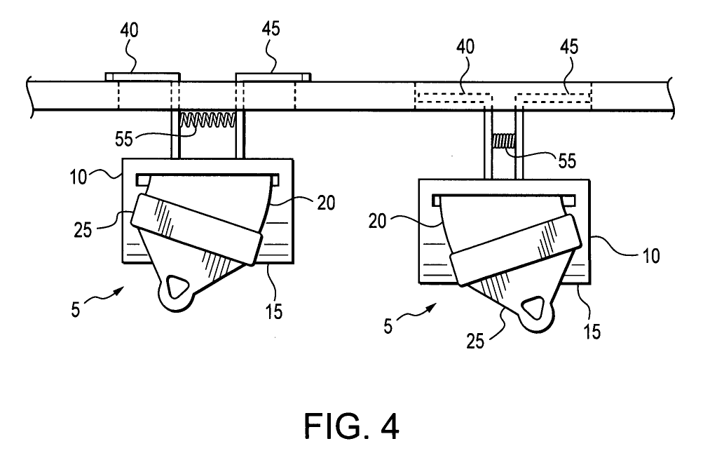

The removably attachable spinal backboard retractable strap device 5, as depicted in FIGS. 1-2, generally comprises a rigid housing 10 with a curved bottom surface 15 containing a strap 20 with a buckle 25 and a spring-loaded clip mechanism 30. In the preferred embodiment, strap 25 is retractable. The housing 10 further comprises a spool 30 upon which the strap 25 is wound. The spool 30 is rotatably secured to an axle in a conventional manner, allowing the spool 30 to revolve around the axle upon conventional bearing surfaces. The housing 10 further comprises conventional means of presenting torque on the spool 30 to cause the strap 25 to be wound onto the spool 30. In the preferred embodiment, the torque is provided with a spring retainer containing a spiral spring mounted in a conventional manner.

The spring loaded clip mechanism 35 is comprised of a pair of parallel squeeze plates 40, 45 slidably set in a track 50 and a coil spring 55 permanently mounted between the parallel squeeze plates, so that the ends of the spring 55 press against the inside surfaces of the squeeze plates 40, 45 and holds the squeeze plates 40, 45 apart.

To removably attach the retractable strap device 5 to a spinal backboard, the user will squeeze the squeeze plates 40, 45 toward each other, compressing the coil spring 55, the user will then pass the squeeze plates through an opening, such as a handhold, in the spinal backboard, and then release the squeeze plates. The squeeze plates 40, 45 will move outwardly under the pressure from the coil spring 55, engaging the inside walls of the opening in the spinal backboard, thus limiting further outward movement of the squeeze plates, and thereby releasably securing the retractable strap device 5 to the spinal backboard.

The squeeze plates 40, 45 will return easily to the at rest positions when the user stops squeezing or exerting force, allowing the squeeze plates to exert pressure against the inside walls of the opening in the spinal backboard, removably securing the strap device to the spinal backboard. The strap device is easily removed by again squeezing the plates together, and pulling the plates downward, disengaging the spinal backboard.

In the preferred embodiment, the squeeze plates 40, 45 are L-shaped, further comprising a leg element oriented at a right angle to the parallel squeeze plates, and the squeeze plates are arranged so that the legs point away from each other and rest on the upper surface of the spinal backboard when in use. The legs are substantially flat and of an appropriate thickness so as to provide as seamless a surface as possible while retaining the necessary thickness to ensure durability and sturdiness. This configuration provides a smooth surface on the top of the spinal backboard while the strap device 5 is in use, and further allows for a more snug and secure fit. In the preferred embodiment, the portion of the squeeze plates that will come into contact with the patient will be covered with rubberized or other material for increased comfort to the patient. This covering can comprise any suitable material to increase the patient's comfort.

The L-shaped squeeze plates can be formed as a single unit, or can be formed of separate elements permanently joined together.

The coil spring 55 is secured between the squeeze plates 40 and 45 so that ends of the spring 55 press against the inside surfaces of the squeeze plates 40 and 45, and holds the squeeze plates apart. While engaged, the squeeze plates 40, 45, move outwardly under the pressure from the coil spring 55 to a position in which the squeeze plates engage the inside walls of the opening in the spinal backboard, thus limiting further outward movement of the squeeze plates. Other alternatives for spring arrangements are available.

The housing 10 and squeeze plates 40, 45 can be constructed of any durable rigid material.

Any other appropriate and effective means for providing the resistance means between the squeeze plates can be employed.

The squeeze plates 35, 40 can be slidably mounted in any effective manner.

The squeeze plates 45, 50 can be constructed of any appropriate sturdy rigid material.

The resistance means between the squeeze plates can be any effective resistance means available.

The strap can be formed of any suitable material. The strap can be formed of weatherproof material. The strap can be provided entirely outside of the strap device housing, without retracting into the strap device housing.

In the preferred embodiment, the straps contain buckles with mating male and female portions at the ends. The straps can be secured about the patient in any effective manner. The straps may be tied together, buckled, or secured with snaps, hook and pile type fasteners, or any other effective means.

This device can be used to secure any medical accessory to a spinal backboard utilizing the spring-loaded clip mechanism. In addition to affixing straps to spinal backboards, there is a distinct need to secure other medical accessories to spinal backboards in a more efficient manner. The curved housing and spring-loaded clip mechanism of the instant invention can removably secure additional medical accessories, such as oxygen equipment, limb fixation devices, splints, and traction devices to the spinal backboard. These additional medical accessories will be removably attached in place of the strap, or in addition to the strap. The additional medical accessories will be attached to the curved housing 10 using any suitable attachment means. The additional medical accessories can be permanently affixed to the inside or outside of the housing 10, or they can be removably attached to the inside or outside of the housing 10 in any conventional manner. Utilizing the same spring-loaded attachment means for the patient straps and other medical accessories provides increased efficiency, safety, ease of use, and cost-effectiveness.

In the preferred embodiment, the squeeze plates are secured so that they cannot be lifted out of the track, and can only move laterally.

In the preferred embodiment, the spring resistance means is concealed within the housing to maintain its integrity and for maximum safety.

The spring resistance means can also be covered in any suitable protective material that allows lateral movement.

The device can further comprise an emergency release button for increased safety, of the type ordinarily used with safety restraint straps.

We have illustrated a preferred embodiment of the invention. However, it is to be understood that changes and variations may be made by those skilled in the art without departing from the spirit of the invention as claimed.

Many variations of the invention will occur to those skilled in the art. All such variations are intended to be within the scope and spirit of the invention. Although some embodiments are shown to include certain features, we specifically contemplate that any feature disclosed in this description may be used together or in combination with any other feature on any embodiment of the invention. We have also contemplated that any feature may be specifically excluded from any embodiment of the invention.

Further aspects of the invention will become apparent from consideration of the drawings and the description of preferred embodiments of the invention. A person skilled in the art will realize that other embodiments of the invention are possible and that the details of the invention can be modified in a number of respects, all without departing from the inventive concept. Thus, the drawings and description are to be regarded as illustrative in nature and not restrictive.

### LIST OF REFERENCE NUMBERS

| | |
|---|---|
| 5 | Removably Attachable Retractable Strap Device |
| 10 | Housing |
| 15 | Curved Bottom Surface |
| 20 | Strap |
| 25 | Buckle |
| 30 | Spool |
| 35 | Spring-Loaded Clip Mechanism |
| 40 | L-shaped Squeeze Plate |
| 45 | L-shaped Squeeze Plate |
| 50 | Track |
| 55 | Coil Spring |

1. A Device comprising:
a. A resilient housing with a curved bottom;
b. A means for attaching a medical accessory to said housing;
c. A means for removably attaching said housing to a patient transport device comprising a pair of parallel spaced squeeze plates set in a track; and
d. A means of resistance biasing the squeeze plates away from each other.

2. The device of claim 1 in which said medical accessory is a flotation device.

3. The device of claim 1 in which said medical accessory comprises oxygen equipment.

4. The device of claim 1 in which said medical accessory is a limb fixation device.

5. The device of claim 1 in which said medical accessory is a traction device.

6. A Patient Transport Strap Device comprising:
a. A resilient housing with a curved bottom;
b. A patient restraint strap;
c. A means for removably attaching said housing to a spinal backboard comprising a pair of parallel spaced squeeze plates set in a track; and
d. A means of resistance biasing the squeeze plates away from each other.

7. A Retractable Strap Device comprising:
a. A retractable strap contained within a resilient housing with a curved bottom surface;
b. A means for removably attaching said housing to a spinal backboard comprising a pair of parallel spaced squeeze plates set in a track; and
c. A means of resistance biasing the squeeze plates away from each other.

8. The device of claim 7 in which the parallel spaced squeeze plates are L-shaped with a leg portion and a foot portion, oriented upside down and inverted with respect to each other so that said leg portions are parallel and spaced apart, and said feet portions point in opposite directions.

9. The device of claim 7 in which the parallel spaced squeeze plates are L-shaped with a leg portion and a foot portion, oriented upside down and inverted with respect to each other so that said leg portions are parallel and spaced apart, said feet portions point in opposite directions, and said feet portions are coated with a resilient coating.

10. The device of claim 7 in which the parallel spaced squeeze plates are L-shaped with a leg portion and a foot portion, oriented upside down and inverted with respect to each other so that said leg portions are parallel and spaced apart, said feet portions point in opposite directions, and said feet portions are covered with a rubberized material.

11. The device of claim 7 further comprising an emergency release button.

12. The device of claim 7 further comprising a means of securing said strap to another strap to restrain a patient comprising male and female mating buckles.

13. The device of claim 7 further comprising a means of securing said strap to another strap to restrain a patient comprising Velcro fasteners.

14. The device of claim 7 further comprising a means of securing said strap to another strap to restrain a patient comprising Snap fasteners.

15. The device of claim 7 further comprising a means of securing said strap to another strap to restrain a patient.

16. The device of claim 7 further comprising a means of selectively restricting further movement of the portion of the retracting strap contained within the strap housing with a catch mechanism to maintain the length of strap secured around the patient.

17. The device of claim 7 in which said means of resistance is a coil spring.

18. The device of claim 7 in which said strap is formed of waterproof material.

19. The device of claim 7 in which said means for removably attaching said housing to a spinal backboard is attached to the top of said resilient housing.

20. The device of claim 7 in which said means for removably attaching said housing to a spinal backboard is attached to the side of said resilient housing.
